(19) United States
(12) Patent Application Publication
(10) Pub. No.: US 2025/0025095 A1
(43) Pub. Date: Jan. 23, 2025

(54) SYSTEM AND METHOD OF DETECTING AND MONITORING NEURODEGENERATIVE AND NEUROLOGICAL DISORDERS

(71) Applicant: KONINKLIJKE PHILIPS N.V., Eindhoven (NL)

(72) Inventors: Junmo An, Cambridge, MA (US); Richard Earl Gregg, Cambridge, MA (US)

(21) Appl. No.: 18/777,755

(22) Filed: Jul. 19, 2024

Related U.S. Application Data
(60) Provisional application No. 63/527,850, filed on Jul. 20, 2023.

(51) Int. Cl. A61B 5/00; A61B 5/0205; A61B 5/11; A61B 5/318

(52) U.S. Cl. CPC A61B 5/4082; A61B 5/0205; A61B 5/1126; A61B 5/318; A61B 5/7264; A61B 2562/0219

(57) ABSTRACT

A system for analyzing a neurological and/or neurodegenerative disorder (ND) is disclosed. The system include an electrocardiogram (ECG) sensor and an inertial measurement unit (IMU) sensor to a subject; a processor; a memory that stores instructions. When executed by the processor, the instructions cause the processor to: gather ECG and IMU data for the subject; input the integrated ECG and IMU data to a first computational model and a second computational model; and infer a presence of the ND based on the first computational model, or a change in the ND based on the second computational model. A method for analyzing and ND is also disclosed.

### CROSS-REFERENCE TO RELATED APPLICATIONS

This patent application claims the priority benefit under 35 U.S.C. § 119(e) of U.S. Provisional Application No. 63/527,850, filed on Jul. 20, 2023, the contents of which are herein incorporated by reference.

### BACKGROUND

There are a number of neurodegenerative disorders and neurological disorders (each referred to herein as an ‘ND’) that affect millions of people around the world. For example, Parkinson's disease (PD), strokes and multiple sclerosis (MS) are common NDs that adversely impact the world's population.

It is important to not only detect the presence of these disorders, but also to monitor changes in a person with an ND so affected over the course of treatment. This monitoring can be done to determine both a decline in a patient's health caused by the progression of the disease as well as improvement in the patient's resulting from medical treatment.

What is needed, therefore, is a method and system for detecting the presence and/or progression of certain NDs in a patient in a reliable way using sensors adapted to gather data from a patient.

### SUMMARY

According to an aspect of the present disclosure, a method of analyzing a neurodegenerative and/or a neurological disorder (ND) is disclosed. The method comprises: applying an electrocardiogram (ECG, also known as EKG) sensor and an inertial measurement unit (IMU) sensor to a subject; gathering ECG data and IMU data for the subject; inputting the integrated ECG data and IMU data to a first computational model and a second computational model; and inferring a presence of the ND based on the first computational model, or a change in the ND based on the second computational model.

According to another aspect of the present disclosure, a system for analyzing an ND is disclosed. The system comprises: an electrocardiogram (ECG) sensor and an inertial measurement unit (IMU) sensor to a subject; a processor; a memory that stores instructions, which when executed by the processor, cause the processor to: gather ECG and IMU data for the subject; input the integrated ECG and IMU data to a first computational model and a second computational model; and infer a presence of the ND based on the first computational model, or a change in the ND based on the second computational model.

According to another aspect of the present disclosure, a tangible, non-transitory computer readable medium that stores instructions is disclosed. When executed by a processor, the instructions cause the processor to: gather ECG and IMU data for a subject; input the ECG and IMU data to a first computational model and a second computational model; and infer a presence of a ND based on a first computational model, or a change in the ND based on a second computational model.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 is a simplified block diagram of a system for imaging a portion of a body, according to a representative embodiment.

FIG. 2A shows spectral analysis of heart rate variability in a healthy subject according to a representative embodiment.

FIG. 2B shows spectral analysis of heart rate variability in a subject having PD according to a representative embodiment.

FIG. 3A illustrates a setup for measuring postural sway using an inertial measurement unit (IMU) sensor on a patient according to a representative embodiment.

FIG. 3B illustrates a setup for measuring postural sway using an IMU sensor in a sway meter in a patient according to a representative embodiment.

FIG. 3C illustrates postural sway traces measured using a force plate during quiet stance in a healthy subject and a person with PD according to a representative embodiment.

FIG. 4 is a graph of elliptical area of postural sway in a group of subjects with PD and a group of healthy subjects while standing quiet with eyes open (EO) and eyes closed (EC), according to a representative embodiment.

FIG. 5A is a flow-chart of a method of data preparation, training, testing, and deploying of machine-learning (ML) and deep-learning (DL) models according to a representative embodiment.

FIG. 5B is a flow-chart of various aspects of the method of training, validating, testing, and deploying of machine-learning (ML) and deep-learning (DL) models in FIG. 5A according to a representative embodiment.

FIG. 6 is a simplified flow-diagram showing a method of detecting a presence of an ND, and the progression or regression of the ND, using computational models according to a representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials, and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms “a,” “an” and “the” are intended to include both singular and plural forms, unless the context clearly dictates otherwise. Additionally, the terms “comprises,” “comprising,” and/or similar terms specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

By the present teachings, a method and system for detecting the presence of an ND, or the change (progression or regression) of an ND, or both, are described. Data from both ECG and IMU sensors are used to train and test computational models used to detect the presence of, or change of NDs of various types, or both, of a subject. Just by way of illustration, in addition to detecting and monitoring the progression/regression of PD, the present teachings are contemplated for the detection and monitoring of other ND conditions including, but not limited, to multiple sclerosis (MS), dementia, Alzheimer's Disease, stroke, brain tumor, neuroinfections, and similar infirmities based on data from ECG and IMU sensors from the subject. The present teachings contemplate sensor-based wearable devices that are portable, and possibly complement various in-home measurements during enabling the monitoring of cardiac health and function, balance and gait stability as well as predict cardiac abnormalities and balanced impairments caused by ND. Furthermore, and among other benefits, the use of both ECG and IMU sensors/data in an integrated system improves the ability to detect the existence and prediction/progression of NDs as compared to ECG or IMU testing alone. In addition, the present teachings provide a practical application of remote testing/monitoring of a subject's ND, for example, at home using a smart phone, a watch/fitness tracker, or computer link, in order to improve detection and monitoring of the change (progression/regression) of the ND. As will become clearer as the present description continues, the ECG and IMU sensors are contemplated to be separate devices connected to the smart phone, watch/fitness tracker, or computer where the methods of the present teachings are carried out. Alternatively, these sensors may be integrated into a smart phone, watch/fitness tracker, or other portable communication device (e.g., a tablet computer or laptop computer). As such, implementation of the methods and systems of the present teachings afford the practical application of remote examination of a subject with ND with on site detection of the existence of the ND and the monitoring of the progression/regression of the ND in the patient in a remote setting. The method and system of the present teachings also improves the function of a computer, and/or smart phone, and/or watch/fitness tracker, by enabling the detecting and monitoring of an ND of a subject remotely, and not requiring testing/monitoring to be done in a hospital or other health facility.

Among other benefits, and as described more fully below, the systems and methods of the present teaching utilize combined/integrated/hybrid ECG and IMU data, which results in superior prediction performance with regard to detecting and monitoring NDs compared to using ECG data or IMU data independently. Specifically, the hybrid approach of a representative embodiment in which ECG and IMU data are combined leads to improved prediction performance for detecting NDs using ML and DL computational models described herein. This integrated method also improves prediction performance for assessing changes in the disease, including disease progression or regression, more effectively and accurately than using ECG or IMU data alone.

The combined data can be processed in two ways. Firstly, the combined data can be processed by utilizing the extracted IMU and ECG features with the ML model, known as feature-based ML. Secondly, the combined data can be processed by utilizing the raw IMU and ECG signals with the DL model, known as end-to-end DL. Both approaches offer distinct advantages and disadvantages, such as those noted below and can be considered for optimal analysis and prediction of NDs. Furthermore, and as will become clearer as the present teachings continue, early detection of certain NDs (e.g., PD, stroke, and MS) is beneficial to establishing proper therapeutic and medicinal interventions, and providing effective treatment/management strategies of NDs. The various embodiments of the present teachings incorporate multimodal ECG/balance biomarkers using an at-home, low-cost portable device (e.g., smartphone) based wearable system, comprising ECG and IMU sensors, will aid in early diagnosis and detection of certain NDs, as well as the severity/progression of the ND and balance impairments in ND patients, for example. This is especially beneficial when the availability of clinical facilities in which to carry out these tests is sparse.

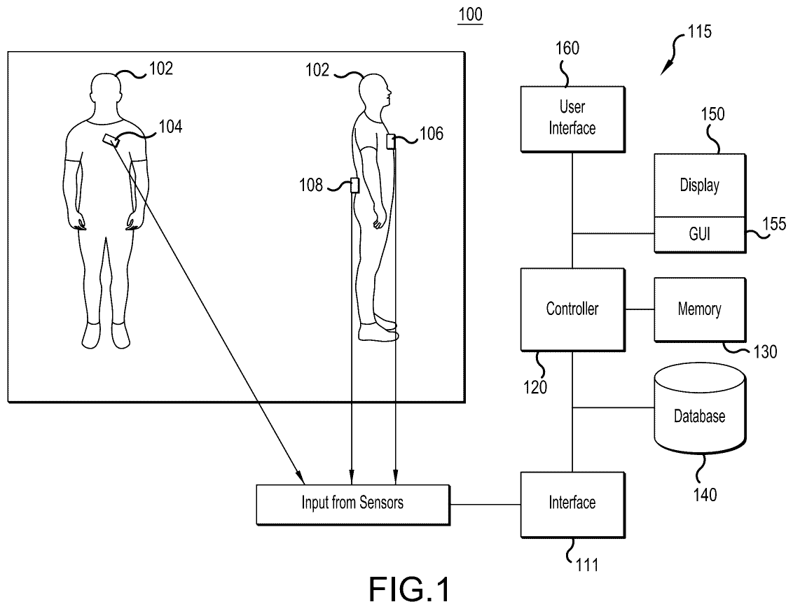

FIG. 1 is a simplified block diagram of a system 100 for detecting a presence and/or monitoring a change of an ND according to a representative embodiment.

Referring to FIG. 1, a system 100 includes sensors 104, 106, 108 worn by a subject 102 and a computer system 115 for detecting and monitoring the ND based on data from the sensors 104, 106, 108.

Sensor 104 is illustratively a combined IMU and ECG sensor, whereas sensors 106 and 108 are an ECG and an IMU sensor, respectively.

IMU sensors contemplated for use in accordance with various representative embodiments are known/commercially available IMU sensors. IMU sensors are devices that measure and report a body's specific force, angular rate, and the orientation of the body, using a combination of accelerometers and gyroscopes. As is known, an accelerometer measures time rate of change of the velocity, whereas a gyroscope measures rotational changes. These IMU sensors can be stand-alone, commercially available devices. Alternatively, the IMU sensors may be included in known devices, such as smart phones, watches/fitness trackers, and portable computers, that include accelerometers and gyroscopes. Accelerometers, for example, are incorporated into smart phones, watches/fitness trackers and portable computers in compass and map applications, tilt sensing, fall sensing, and games requiring motion sensing. Similarly, gyroscopes are found in known consumer electronics (smart phones, watches/fitness trackers, and portable computers) that incorporate microelectromechanical system (MEMS) gyroscopes. As described more fully herein, data from IMU's include, but are not limited to, subject displacement, velocity, and postural sway, comprising the elliptical area of postural sway, root mean square of postural sway (RMS sway/distance), entropy of postural sway, fractal dimension of postural sway, fractal dynamics of postural sway, a Lyapunov exponent of postural sway, power spectral density (PSD) of postural sway, and others similar measures.

ECG sensors contemplated for use in accordance with various representative embodiments are known/commercially available ECG sensors that are also adapted for remote measurements and can be stand-alone sensors, or can be incorporated into smart phones, watches/fitness trackers, and portable communication devices/computers and computers. As described more fully herein, the data from the ECG sensors of various representative embodiments include, but are not limited to, raw ECG signals, heart rate variability (HRV), QT-interval, power spectral density (PSD), and others.

Notably, the inclusion of ECG and IMU functionality in a watch, such as a fitness tracker, and other portable communications devices are contemplated, and enable the gathering of data by the following illustrative method. To gather ECG data, the watch is on one arm or the other portable communication device (e.g., smart phone) is in one hand. The subject maintains the watch/other portable communication device with both hands at chest level, and places a finger from the other hand on the ECG electrode on the watch/other portable communication device to record the ECG data. To gather IMU data, for reasons described more fully below, the watch/portable communication device are held in the region of the L4/L5 vertebral level (i.e., the lower back). To obtain simultaneous ECG and IMU data, the watch is on one arm or the other portable communication device (e.g., smart phone) is in one hand. The subject stands up straight and maintains the watch/other portable communication device with both hands at chest level or in the region of the L4/L5 vertebral level (i.e., the lower back), and places a finger from the other hand on the ECG electrode on the watch/other portable communication device to record simultaneous ECG and IMU data.

In accordance with representative embodiments described below, data from IMU and ECG sensors are used to construct a first computational model that is adapted to predict an existence of an ND. The data from the IMU and ECG sensors are also used to construct a second computational model to measure the change (e.g., progression or regression) of the ND over time. This second computational model is thus useful to monitor changes in a stage of the ND (e.g., the stage of PD in a Parkinson's patient). These changes may be for the worse, or for the better, and thus can be used as a guide to measure the effectiveness of treatment of the ND. As described more fully below, the first and second computational models are a feature-based machine learning (ML) model or an end-to-end deep learning (DL) model, or a combination of both, and are trained using data from IMU and ECG sensors. Stated somewhat differently, both ML and DL models may be used in accordance with certain representative embodiments, or either ML or DL models may be used in accordance with certain representative embodiments. The determination of whether to use an ML or DL computational model may be made by consideration of certain advantages/disadvantages associated with each type of model, such as those noted below.

The first and second computational models of the representative embodiments are stored as executable instructions (executable computer code) on a tangible, non-transitory computer readable medium, such as described below. As described below, execution of the instructions comprising the first computational model by a processor or similar computer device cause the processor or similar computer device to infer a presence of the ND. Similarly, execution of the instructions comprising the second computational model by a processor or similar computer device cause the processor or similar computer device to infer a change in the ND. The first computational model may be referred to herein as a classification model, with the system 100 functioning in a detection mode; and the second computational model may be referred to herein as a regression model, with the system functions in a serial comparison mode.

The computer system 115 receives IMU and ECG data from the ECG sensor 106 and the IMU sensor 108, or from the combined IMU/ECG sensor 104 and stores and processes the IMU and ECG data according to the embodiments discussed herein. The computer system 115 includes a controller 120, a memory 130, a database 140 and a display 150.

The controller 120 interfaces with the sensors 104˜106 through an imaging interface 111. The memory 130 stores instructions executable by the controller 120. When executed, and as described more fully below, the instructions cause the controller 120 to implement processes that include inferring a presence of the ND based on the first computational model, or a change in the ND based on the second computational model. In addition, the controller 120 may implement additional operations based on executing instructions, such as instructing or otherwise communicating with another element of the computer system 115, including the database 140 and the display 150, to perform one or more of the above-noted processes.

The controller 120 is representative of one or more processing devices, and is configured to execute software instructions to perform functions as described in the various embodiments herein. The controller 120 may be implemented by field programmable gate arrays (FPGAs), application specific integrated circuits (ASICs), a general purpose computer, a central processing unit, a computer processor, a graphics processing unit (GPU), a microprocessor, a microcontroller, a state machine, programmable logic device, or combinations thereof, using any combination of hardware, software, firmware, hard-wired logic circuits, or combinations thereof. Additionally, any processing unit or processor herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems, such as in a cloud-based or other multi-site application. The term computing device should also be interpreted to include a collection or network of computing devices each including a processor or processors. Programs have software instructions performed by one or multiple processors that may be within the same computing device or which may be distributed across multiple computing devices.

The memory 130 may include a main memory and/or a static memory, where such memories may communicate with each other and the controller 120 via one or more buses. The memory 130 stores instructions used to implement some or all aspects of methods and processes described herein. The memory 130 may be implemented by any number, type and combination of random access memory (RAM) and read-only memory (ROM), for example, and may store various types of information, such as software algorithms, which serves as instructions, which when executed by a processor cause the processor to perform various steps and methods according to the present teachings. For example, in accordance with various representative embodiments, the memory 130 that stores instructions, which when executed by the processor, cause the processor to prepare the ECG and IMU data for use in the computational models by performing preprocessing, feature extraction, normalization and integrated into a suitable format for ML or DL training to train the first and second computational models. The feature-based ML models typically extract relevant features from the data and use them as inputs for classification or regression tasks. In contrast, the end-to-end DL models learn directly from the raw data, bypassing the need for explicit feature extraction. After training, the first and second computational models are subjected to testing to evaluate their performance and ensure their effectiveness in classifying or predicting outcomes accurately. Finally, the trained and tested models (also called predictive models) are deployed, making them available for real-world applications. This enables the feature-based ML and end-to-end DL models to perform their intended tasks, such as classification or regression, using both ECG and IMU data, either on a server or potentially on a smartphone as noted above.

More generally, although not necessarily, the data preparation including preprocessing, feature extraction, normalization, and integrated into a suitable format for ML or DL models to train and test the first and second computational models, as well as execution of the first and second computational models may be done using another processor and memory that are not necessarily part of the computer system 115 or the system 100. In this case, after being trained, the computational model may be stored as executable instructions in memory 130, for example, to be executed by a processor of the controller 120. Furthermore, updates to the first and second computational models may also be provided to the computer system 115 and stored in memory 130. Finally, and as will be apparent to one of ordinary skill in the art having the benefit of the present disclosure, according to a representative embodiment, the first and second computational models may be stored in a memory and executed by a processor that are not part of the computer system 115, but rather is connected to the IMU and ECG sensors 104-108 through an external link (e.g., a known type of internet connection). Just by way of illustration, the computational model may be stored as executable instructions in a memory, and executed by a server that is remote from the subject and IMU and ECG sensors 104-108. When executed by the processor in the remote server, the instructions cause the processor to effect data preparation including preprocessing, feature extraction, normalization, and integrated into a suitable format for ML or DL models to train and test the first and second computational models, as well as execute the first and second computational models.

The various types of ROM and RAM may include any number, type and combination of computer readable storage media, such as a disk drive, flash memory, an electrically programmable read-only memory (EPROM), an electrically erasable and programmable read only memory (EEPROM), registers, a hard disk, a removable disk, tape, compact disk read only memory (CD-ROM), digital versatile disk (DVD), floppy disk, Blu-ray disk, a universal serial bus (USB) drive, or any other form of storage medium known in the art. The memory 130 is a tangible storage medium for storing data and executable software instructions, and is non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The memory 130 may store software instructions and/or computer readable code that enable performance of various functions. The memory 130 may be secure and/or encrypted, or unsecure and/or unencrypted.

Similarly, the database 140 stores data and executable instructions used to implement some or all aspects of methods and processes described herein. As will be described more fully below, the database 140 illustratively stores ECG and IMU data from current and past measurements, such as ground truth data (GTD).

The database 140 may be implemented by any number, type and combination of RAM and ROM, for example, and may store various types of information, such as software algorithms, Al models including machine learning and deep learning models, and computer programs, all of which are executable by the controller 120. The various types of ROM and RAM may include any number, type and combination of computer readable storage media, such as a disk drive, flash memory, EPROM, EEPROM, registers, a hard disk, a removable disk, tape, CD-ROM, DVD, floppy disk, Blu-ray disk, USB drive, or any other form of storage medium known in the art. The database 140 is a tangible storage medium for storing data and executable software instructions that are non-transitory during the time software instructions are stored therein. The database 140 may be secure and/or encrypted, or unsecure and/or unencrypted.

“Memory” and “database” are examples of computer-readable storage media, and should be interpreted as possibly being multiple memories or databases. The memory or database may for instance be multiple memories or databases local to the computer, and/or distributed amongst multiple computer systems or computing devices.

The controller 120 illustratively includes or has access to an AI engine, which may be implemented as software that provides artificial intelligence and applies ML and DL described herein. The AI engine, which provides the first and second computational models described below, may reside in any of various components in addition to or other than the controller 120, such as the memory 130, the database 140, an external server, and/or a cloud, for example. When the AI engine is implemented in a cloud, such as at a data center, for example, the AI engine may be connected to the controller 120 via the internet using one or more wired and/or wireless connection(s). The artificial intelligence (AI) engine may be connected to multiple different computers including the controller 120, so that the artificial intelligence and machine-learning described below in connection with various representative embodiments are performed centrally based on and for a relatively large set of medical facilities and corresponding subjects at different locations. Alternatively, the AI engine may implement the machine learning and the deep learning locally to the controller 120, such as at a single medical facility or in conjunction with sensors 104˜108, which may be components of a portable communication device (e.g., a smart phone, or watch/fitness tracker).

The interface 160 may include a user and/or network interface for providing information and data output by the controller 120 and/or the memory 130 to the user and/or for receiving information and data input by the user. That is, the interface 160 enables the user to enter data and to control or manipulate aspects of the processes described herein, and also enables the controller 120 to indicate the effects of the user's control or manipulation. The interface 160 may include one or more of ports, disk drives, wireless antennas, or other types of receiver circuitry. The interface 160 may further connect one or more user interfaces, such as a mouse, a keyboard, a mouse, a trackball, a joystick, a microphone, a video camera, a touchpad, a touchscreen, voice or gesture recognition captured by a microphone or video camera, for example.

The display 150 may be a monitor such as a computer monitor, a television, a liquid crystal display (LCD), a light emitting diode (LED) display, a flat panel display, a solid-state display, or a cathode ray tube (CRT) display, or an electronic whiteboard, for example. The display 150 may also provide a graphical user interface (GUI) 155 for displaying and receiving information to and from the user.

As alluded to above, and as described in more detail below, in accordance with various representative embodiments, detection of NDs, such as PD, stroke, MS, and similar infirmities, based on ECG and IMU data is inferred using a first computational model; and progression/regression of NDs, such as PD, stroke, MS, and similar infirmities, based on ECG and IMU data is inferred using a second computational model. Notably, in accordance with representative embodiments, the development of the first and second computational models is part of the development of the system 100. The first and second computational models are provided as fully trained and tested models as software (executable computer code/instructions) stored in memory, and are executed by a processor of the computer system 115 as described more fully below.

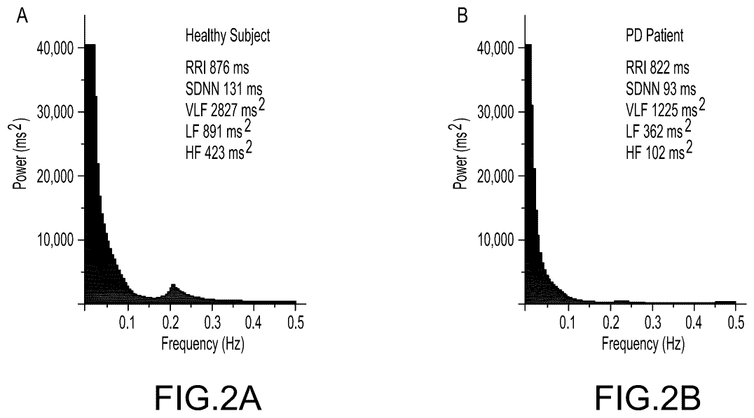

FIG. 2A shows spectral analysis of heart rate variability in a healthy subject. The data used to provide the heart rate variability is derived from ECG data gathered from a subject using an ECG sensor as described above.

Referring to FIG. 2A, a graph of power spectrum of heart rate variability (HRV) of ECG is depicted from a healthy subject. Power spectral analysis of HRV is a quantitative method used to examine the distribution of power across different frequency components within the variation in time intervals between consecutive heartbeats. The HRV of ECG provides insights into the autonomic nervous system's influence on heart rate regulation. PSD of HRV is a valuable tool for screening autonomic dysfunctions in ND patients. Notably, the PSD of HRV differs significantly between healthy adults and patients with PD. The power levels within the HRV PSD exhibit notable variations across different severities of PD. Further details relating to FIGS. 2A and 2B can be found in “Ambulatory ECG and Analysis of Heart Rate Variability in Parkinson's Disease” to Haapaniemi, T. H., Pursiainen, V., Korpelainen, J. T., Huikuri, H. V., Sotaniemi, K. A., & Myllylä, V. V. (Journal of neurology, neurosurgery & psychiatry, 70(3), 305-310 (2001), the disclosure of which is specifically incorporated herein by reference and for all purposes.

FIG. 2B shows spectral analysis of heart rate variability (HRV) in a subject having PD. The data used to provide the heart rate variability is derived from ECG data gathered from a subject using an ECG sensor as described above.

Referring to FIG. 2B, a graph of power spectrum of HRV of ECG is depicted from a PD subject.

As described more fully below, the HRV data (or other data, or both) gathered from the ECG sensor are used in many aspects of the present teachings. For example, the HRV data from a number of subjects may be used as GTD in training and testing an ML model used to infer a presence of the ND based on the first computational (ML) model, or a change in the ND based on the second computational (ML) model. Feature-based ML models first extract features from the GTD, and then using these features to train and test a model.

Alternatively, these data may be used to train an end-to-end DL model for each procedure in a patient. End-to-end DL models learn to map the input data directly to the output data without the need for explicitly extracting features. With the integration of ECG and IMU data, these models automatically learn pertinent features, optimizing the learning process and enhancing accuracy.

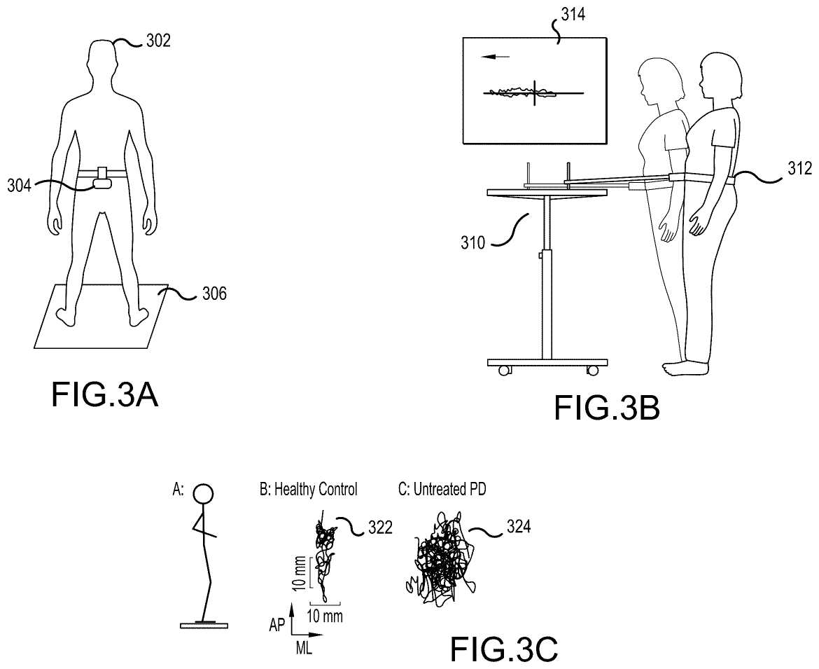

FIGS. 3A and 3B show setups for gathering data useful in training the ML and DL computational models of various representative embodiments. These setups are provided merely for illustrating ways to gather data and the types of data useful to the various representative embodiments, and are not intended to be limiting. Rather, similar IMU data may be gathered using known portable and wearable IMU sensors.

FIG. 3A illustrates a setup for measuring sway using an IMU sensor 304 on a patient according to a representative embodiment. The IMU sensor 304 is located in the lower back region of a subject 302, which is a common place to locate an IMU sensor 304. Further details of the measurement setup of FIG. 3A can be found in “Assessment of Postural Sway in Individuals with Multiple Sclerosis using a Novel Wearable Inertial Sensor” to Sun, R., Moon, Y., McGinnis, R. S., Seagers, K., Motl, R. W., Sheth, N., and Sosnoff. J. J. Digital biomarkers, 2(1), 1-10 (2018)), the disclosure of which is specifically incorporated herein by reference and for all purposes.

The placement of an IMU sensor in the lower back, particularly at the L4/L5 vertebral level, is a common choice for several reasons. The lower back, including the L4/L5 vertebra, is an integral part of the trunk of a human body and is close to the body's center of mass (COM). Also, placing the sensor at the L4/L5 vertebra minimizes interference with the body's natural movements (e.g., caused by breathing). Further details of the placement of an IMU sensor on the lower back of a subject may be found in “Wearable Inertial Sensors to Assess Standing Balance: A systematic review” to Ghislieri, M., Gastaldi, L., Pastorelli, S., Tadano, S., and Agostini, V. Sensors, 19(19), 4075 (2019), the disclosure of which is specifically incorporated herein by reference and for all purposes.

The subject 302 is disposed on a force plate 306, and data regarding motion of the subject is gathered by the IMU sensor 304 and the force plate 306. Data gathered by the IMU sensor 304 and the force plate 360 may include postural sway. Notably, postural sway may include elliptical area of postural sway, RMS of postural sway, entropy of postural sway, fractal dimension of postural sway, fractal dynamics of postural sway, Lyapunov exponent of postural sway, and power spectral density of postural sway, for example, and may be gathered and used in the inference of detection of an ND, or a progression/regression of an ND, or a combination thereof.

FIG. 3B illustrates a setup for measuring sway using an IMU sensor 312 in a sway meter 310 in a patient according to a representative embodiment. The movement or sway for the test is shown at 314.

More generally, and among other aspects and characteristics, the IMU sensors of the various representative embodiments (e.g., the IMU sensors described in connection with FIG. 1) measure fluctuations in the center of pressure (COP) instead of using a sway meter or force plate such as shown in FIGS. 3A and 3B. Beneficially, these portable IMU sensors contemplated for use in accordance with the present teachings are comparatively small, lightweight, and portable devices that can be worn on the body to measure acceleration, rotation, and orientation. These IMU sensors can be used to track postural sway by measuring the small changes in the body's center of mass while an individual is in a standing position.

FIG. 3C depicts the postural sway traces in Anterior-Posterior (AP) and Medio-Lateral (ML) directions of both healthy adults and PD patients standing on a force plate. Again, the force plate is utilized to measure the fluctuations in the COP, providing insights into postural sway. Notably, similar data may be gathered using a known portable/wearable IMU sensor in accordance with a representative embodiment. As such, using portable/wearable IMU sensors also provides a reliable and valid measurement of postural sway. This method offers advantages over other approaches, such as using a force plate or sway meter, which are both costly and limited to laboratory settings. Further details of FIG. 3C may be found in “I Sway: A Sensitive, Valid and Reliable Measure of Postural Control” to Mancini, M., Salarian, A., Carlson-Kuhta, P., Zampieri, C., King, L., Chiari, L., & Horak, F. B. Journal of Neuroengineering and Rehabilitation, 9, 1-8 (2012). Additional details of FIG. 3C may also be found in “Framework for Understanding Balance Dysfunction in Parkinson's Disease” Schoneburg, B., Mancini, M., Horak, F., & Nutt, J. G. Movement Disorders” 28(11), 1474-1482 (2013). The disclosures of these documents are specifically incorporated herein by reference and for all purposes.

As described more fully below, the IMU data such as postural sway (or other data, or both) gathered from the IMU sensors 304, 312 are used in many aspects of the present teachings. Postural sway data can be derived from IMU data, which includes accelerometer and gyroscope readings, by employing a fusion sensor algorithm. Examples of fusion sensor algorithms include the Kalman filter, Complementary filter, Madgwick/Mahony filter, quaternion-based complementary filter, among others. These algorithms combine the data from the accelerometer and gyroscope sensors to generate a more precise estimation of the user's postural sway. By utilizing both sensors, the accelerometer and gyroscope can compensate for each other's noise and drift errors, resulting in enhanced movement tracking with improved completeness and accuracy. For example, the postural sway data from a number of subjects may be used as GTD in training an ML model used to infer a presence of the ND based on the first computational (ML) model, or to infer a change in the ND based on the second computational (ML) model. Feature-based ML models first extract features from the GTD, and then apply these features to train and test a model.

Alternatively, in accordance with a representative embodiment, the postural sway data may be used to train an end-to-end DL model for each procedure in a patient. End-to-end DL models learn to map the input data directly to the output data without explicitly extracting features.

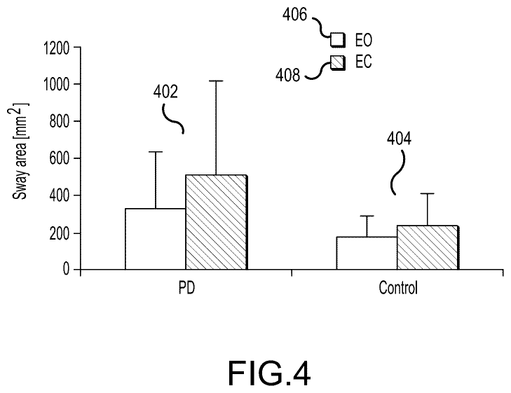

FIG. 4 is a graph of elliptical area of postural sway including standard deviation in a group of subjects with PD 404 and a group of healthy subjects 402 while standing quiet with eyes open (EO) 406 and eyes closed (EC) 408, according to a representative embodiment. These data may be gathered using a combined ECG/IMU sensor 104, or IMU sensor 108.

Again, other IMU data are contemplated for detection and progression/regression of NDs. These include, but are not limited to, elliptical area of postural sway, root mean square of postural sway, entropy of postural sway, fractal dimension of postural sway, fractal dynamics of postural sway, Lyapunov exponent of postural sway, power spectral density of postural sway, and similar measures.

As described more fully below, the IMU data, such as elliptical area of postural sway, gathered from the IMU sensors to provide the data of FIG. 4 are used in many aspects of the present teachings. For example, the elliptical area of postural sway data from a number of subjects may be used as GTD in training an ML model used to infer a presence of the ND based on the first computational (ML) model, or a change in the ND based on the second computational (ML) model. Feature-based ML models first extract features from the GTD, and then using these features to train a model.

Alternatively, these data may be used to train an end-to-end DL model for each procedure in a patient. End-to-end DL models learn to map the input data directly to the output data without explicitly extracting features.

As noted to above, detection and monitoring of the progression/regression of certain NDs are inferred using first computational model (sometimes referred to as a classification model for detection) and a second computational model (sometimes referred to as a regression model for serial comparison), as mentioned above. Both ML and DL computational models are contemplated for use in connection with the systems and methods of the various representative embodiments.

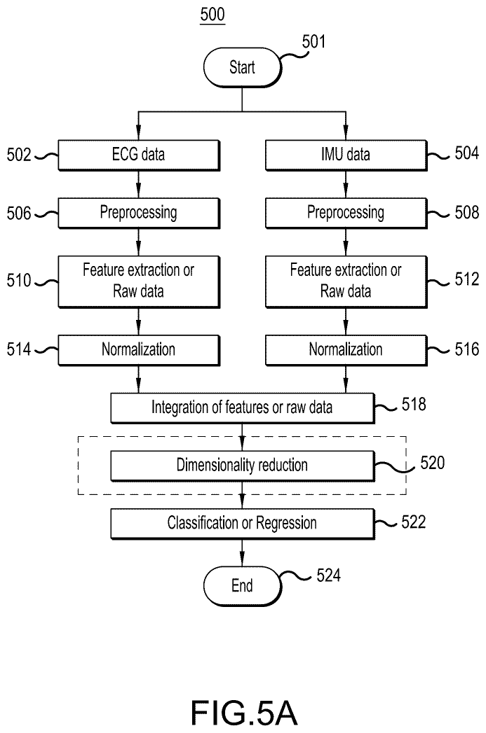

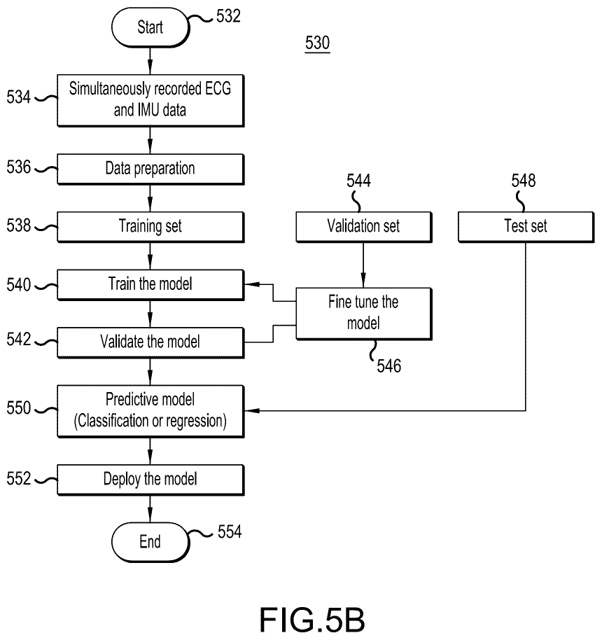

Both feature-based ML and DL computational models are trained. The training sequence of each of the first and second computational models is carried out using one of a number of machine-learning techniques known to those of ordinary skill in the art of AI and mathematical models. These are described in connection with various representative embodiments in connection with FIGS. 5A and 5B. In machine-learning, an algorithmic model “learns” how to transform its input data into meaningful output. During the learning sequence, the computational model is shown known examples of inputs and their correct or desired output.

Training of feature-based ML computational models includes the use of so-called ground truth data (GTD), as mentioned above, which comprises data related to certain parameters (sometimes referred to as “features”) germane to the particular goal of the ML. GTD are generally gathered from a comparatively large sample of sources, examples of which are described below for the ML computational models in connection with various representative embodiments. During the learning sequence, the computational model adjusts its inner parameters given the input parameters of the examples, and produces the corresponding meaningful output or so-called target. The adjustment process is guided by instructions on how to measure the distance between the currently produced output and the desired output. These instructions are called the objective function.

Feature-based ML computational models work by first extracting features from the data, and then using those features to train a model. Feature extraction is a process in ML and signal processing that involves selecting and extracting relevant features (i.e., characteristics or attributes) from raw signal data (e.g., ECG, IMU, etc.). The goal of feature extraction is to transform the original data into a more compact and informative representation that can be used by an ML algorithm to improve its performance on a specific task. To get a better understanding of the topic, feature-based ML involves manually extracting features from signal data, and then using a ML algorithm to learn a model from those features. For example, according to various representative embodiments features, such as HRV, QTc interval, and PSD, can be extracted from ECG data, while velocity, path length, RMS, entropy, elliptical area, etc. of postural sway can be extracted from IMU data. GTD related to these features are used to train and test the ML computational model to detect the presence of an ND and to measure the change (progression/regression) of the stage of an ND.

Once trained and tested, the feature-based ML computational models of various representative embodiments are algorithms (executable computer code/instructions) stored (e.g., in database 140 or memory 130) and executed by a processor (e.g., a processor in the controller 120).

By contrast to feature-based ML computational models, end-to-end DL models learn to map the input data directly to the output data without explicitly extracting features. Continuing with the illustrative data as an example, according to various representative embodiments, these input data comprise raw ECG and raw IMU data. These raw data are then used to train and test the model. In DL computational models, which are a subset of ML computational models, such as those of the representative embodiments, the inner parameters of the computational model are organized into successively connected layers, where each layer produces increasingly meaningful output as input to the next layer, until the last layer which produces the final output. Deep learning layers are typically implemented as so-called neural networks, that is, layers are comprised of a set of nodes, each representing an output value and a prescription on how to compute this output value from the set of output values of the previous layer's nodes. Because the prescription is a weighted sum of transformed output values of the previous layer's nodes, each node only needs to store the weights. The transformation function is the same for all nodes in a layer and is also called activation function. There are a limited number of activation functions that are used today. A particular way to set which previous layer's nodes provide input to a next layer's node is convolution. Networks based on this way are called convolutional neural networks (CNN).

Thus, in the learning phase or so-called training, for each example, the output of the final layer is computed. Outputs for all examples are compared with the desired outputs by way of the objective function. The output of the objective function, the so-called loss, is used as a feedback signal to adjust the weights such that the loss is reduced. The adjustment, i.e. which weights to change and by how much, is computed by the central algorithm of deep learning, so-called backpropagation, which is based on the fact that the weighted sums that connect the layer nodes are functions that have simple derivatives. The adjustment is iterated until the loss reaches a prescribed threshold or no longer changes significantly.

A deep learning neural network thus can be stored (e.g., in database 140 or memory 130) as a topology that describes the layers and activation functions and a (large) set of weights (simply values). A trained neural network is the same, only the weights are now fixed to particular values. Once the neural network is trained, it is put to use, that is, to predict output for new input for which the desired output is unknown.

Notably, the weights or coefficients in ML or DL models are essential parameters that are learned during the training process. Typically, as a ML/DL model becomes more complex, the size of its weights increases. This is primarily due to the fact that more intricate models require learning a greater number of relationships between input and output data. The size of the weights in a ML/DL model is influenced by various factors, including the model's architecture, connectivity, data dimensionality, model capacity, any regularization techniques, etc. employed during training. Consequently, it is challenging to determine the exact number of weights in advance.

Finally, and as alluded to above, there are advantages and drawbacks to both feature-extracted ML and end-to-end DL computational models contemplated for use in connection with various representative embodiments. These advantages and drawbacks are considered in the determination of which type of model to use.

Generally ML computational models are more easily understood and interpreted compared to DL computational models. However, ML computational models can be less accurate in their predictions/inferences than DL computational models. Specifically, compared to DL computational models, ML computational models can be less accurate when the GTD are more complex or noisy. As such, when interpretability of a model's output is more important than its accuracy, it is beneficial to implement an ML computational model over a DL computational model. By contrast, when accuracy of a model's output is more important than its interpretability, it is beneficial to implement a DL computational model over an ML computational model.

Other considerations to be weighed in the decision of whether a DL model or ML model should be implemented in connection with the various representative embodiments include the data requirement for training the model. By comparison, ML computational models require comparatively less data for training than DL computational models. As such, when the availability of data for training the model is comparatively low, and the complexity of problem being solved by the model is of a lesser degree, the selection of an ML to compute the detection or progression/regression of a ND may be the best choice. By contrast, when the availability of data for training the model is comparatively high, and the complexity of problem being solved by the model is of a greater degree, the selection of a DL to compute the detection or progression/regression of an ML may be the best choice.

FIG. 5A is a flow-chart of a method 500 of data preparation, training, validating, testing, and deploying of ML and DL models according to a representative embodiment. Various aspects and details of the method 500 may be common to those described above in connection with the representative embodiments of FIGS. 1-4. These aspects and details may not be repeated in order to avoid obscuring the presently described representative embodiments. It is emphasized that the method 500 is adapted to be carried out using a computer system such as computer system 115, or may be carried out on another computer system, with the trained and tested ML or DL models being stored as executable code (instructions) in memory 130 or database 140 for execution by a processor of the controller 120. Alternatively, the trained and tested ML or DL models may be stored remotely and executed remotely as well.

At 501 the method starts.

At 502 and 504, the method continues with the gathering of a multimodality data set (i.e., ECG and IMU data) from healthy adults and patients with an ND (e.g., PD, stroke, dementia or MS) using both ECG and IMU sensors such as described above. Notably, matching the age and gender of both healthy adults and patients in a particular study beneficially may result in higher accuracy compared to non-matching.

At 502, the method comprises collecting ECG data using an ECG sensor. Notably, the data gathered at 502 may be used as GTD in an ML model, or as raw data in a DL model. These data may be taken from a comparatively large number of subjects and stored in the computer system 115 for use in training and testing the first and second computational models.

At 504, the method comprises collecting of IMU data using an IMU sensor. These data include, for example, postural sway, comprising one or more of elliptical area of postural sway, RMS of postural sway, entropy of postural sway, fractal dimension of postural sway, fractal dynamics of postural sway, Lyapunov exponent of postural sway, and power spectral density of postural sway, for example. Notably, the data gathered at 502 may be used as GTD in an ML model, or as raw data in a DL model. These data may be taken from a comparatively large number of subjects and stored in the computer system 115 for use in training and testing the selected model.

At 506 the method comprises preprocessing of the ECG data collected at 502, and at 508, the method comprises preprocessing of the IMU data collected at 504. Preprocessing comprises removing undesirable data including, for example, removing noise, artifacts, and baseline wander from the data collected at 502, 504. At 510, the method comprises feature extraction or raw data extraction of ECG data that has been preprocessed. The feature extraction may be carried out using a number of known techniques, including, but not limited to, random forest (RF), recursive feature elimination (RFE), and Boruta methods. RF, RFE and Boruta are known methods utilized for measuring feature importance rather than feature extraction. In the feature extraction step (at 510 and 512 described below), specific features such as HRV, QTc interval (QTc or QTcB), and PSD are extracted from the ECG data. Similarly, features related to postural sway, including displacement, velocity, path length, PSD, RMS, elliptical area (sway area), entropy, fractal dimension, and Lyapunov exponent, are extracted from the IMU data.

As noted above, feature extraction is carried out for training an ML computational model of a representative embodiment, and raw data extraction is carried out for training a DL computational model of representative embodiments. These data are the data being used in a particular test of a subject by applying the model to the data from the subject. As such, the extracted features are germane to the particular ND being tested. Just by way of illustration, ECG signals from the ECG sensor are processed to calculate heart rate, average heart rate, HRV and QT interval. In a test to detect and monitor PD, the extracted features from the ECG signals may include a reduced heart rate variability, a prolonged QT interval, and/or an increased QT dispersion, for example. Extracted features based on the measured ECG signals for a stroke may include atrial fibrillation, left ventricular hypertrophy, and ST segment changes, whereas extracted features for MS may include sinus bradycardia, atrioventricular block, and bundle branch block.

At 512, the method comprises feature extraction or raw data of IMU data that has been preprocessed. Again, the feature selection may be carried out using a number of known techniques, including, but not limited to random forest (RF), recursive feature elimination (RFE), and Boruta methods described above.

As noted above, feature extraction is carried out for training an ML computational model of a representative embodiment, and raw data extraction is carried out for training a DL computational model of a representative embodiment. Again, these data are the data being used in a particular test of a subject by applying the model to the data from the subject. As such, the extracted features and/or raw data are germane to the particular ND being tested. Just by way of illustration, IMU signals from the IMU sensor are processed to calculate displacement, velocity, path length, and postural sway, including elliptical area, RMS, entropy, fractal dimension, fractal dynamics, Lyapunov exponent, PSD of the postural sway.

At 514 and 516, the extracted ECG and IMU data, respectively, are normalized. As is known, normalization of data is carried out to avoid errors during model training caused by comparatively large magnitude data. Data normalization may be done, for example, by calculating the mean and standard deviation of the data, which are then used in techniques such as min-max normalization, Z-score normalization, and other known techniques. These values are multiplied by a gain so that the resulting standard deviation is equal to one. In this way, all data have a mean of zero, and the standard deviation of one. Beneficially, normalizing a signal prevents the algorithm of the computational model from assigning too much weight to any one piece of data.

At 518, the features of both the raw ECG and IMU data are integrated or concatenated. ECG signals may be represented as one-dimensional (1D) vectors when using a single-lead ECG sensor. However, when a multi-lead ECG sensor is used, ECG signals can be represented as a two-dimensional (2D) array, where each row represents a lead and each column represents a sample or vice versa. By contrast, postural sway signals obtained from IMU sensor using a fusion sensor algorithm mentioned above are typically represented as three-dimensional (3D) vectors, consisting of the x, y, and z axes multiplied by the number of samples. In accordance with a representative embodiment, only the signals from the x and y axes are utilized, while the z-axis signal is disregarded. Notably, the specific naming of the axes may vary depending on the sensor and its pose or orientation. There are several methods available to combine or integrate the raw ECG signals with the postural sway signals obtained from the IMU. These methods are known and comprise concatenation, stacking, fusion, among other methods. The specific details of how to combine/integrate the ECG and IMU data are within the purview of one of ordinary skill in the art. It is further noted that in keeping with a representative embodiment, ECG and IMU data can be combined/integrated together.

At 520 reduction of the dimensionality of the data is optionally carried out. Notably, this step is shown as surrounded by a dashed line, and can be deleted from the method 500 if desired. As is known. dimensionality reduction is a technique that is used to reduce the number of features in a dataset. This can be beneficial for a number of reasons, including: improved accuracy, reduced training time, overfitting prevention, and improved visualization and interpretability. Dimensionality reduction may be carried out using a known method including, but not limited to principal component analysis (PCA), multidimensional scaling (MDS), locally linear embedding (LLE), isomap embedding, t-distributed stochastic neighbor Embedding (t-SNE), among other known methods.

At 522, in accordance with one aspect of the method 500, the method 500 may continue with the application of an ML algorithm to assign the integrated features to a class as being normal and abnormal. Illustrative ML algorithms include, but are not limited to multi-layer perceptron network (MLP), logistic regression, k-nearest neighbors (kNN), radial basis function kernel support vector machines (RBF SVM), gaussian naive bayes, gaussian process, decision tree, random forest, stochastic gradient descent (SGD), and quadratic discriminant analysis (QDA). Notably, logistic regression model is a statistical model that predicts the probability of an event or outcome belonging to one of two classes, and as such may be used for binary classification. Despite its name containing “regression,” logistic regression is a classification algorithm rather than a regression algorithm.

Just by way of illustration, a comparatively simple ML algorithm, logical regression, may be applied at 522. In a logical regression, a weight is assigned for each of the selected features used in the ML model. The algorithm then multiplies the features by the respective weight and sums the results using a sigmoid function, for example. The result provides the output in a range from a minimum value (e.g., zero) to a maximum value (e.g., one). A value would then be set above which the result (e.g., 0.5) is deemed abnormal and below which is deemed normal.

When “Detection” mode (at 612) is selected, “Classification” (at 522) will be used to detect ND. When “Serial Comparison” mode (at 614) is selected, “Regression” (at 522) will be used to predict changes in ND conditions for serial comparison. The purpose of “Serial Comparison” mode is thus used to track and forecast the progression or regression of ND conditions of a particular subject over time, allowing for comparisons between different points in time.

At 522, in accordance with one aspect of the method 500, the method 500 continues with the application of an end-to-end DL model to classify the integrated ECG and IMU data as either normal or abnormal (referred to herein to as classification), and to predict the changes in ND conditions (e.g., the stage of the ND). Illustrative DL algorithms include, but are not limited to LSTM, AlexNet, SqueezeNet, GoogLeNet, ShuffleNet, MobileNet, VGG, ResNet, SE-ResNet, ResNeXt, DenseNet, SENet, Inception, NASNet, Transformer DL algorithms, among other known algorithms. Notably, in accordance with a representative embodiment a sigmoid layer and binary cross-entropy loss may be used. Alternatively, other loss functions can also be used depending on the specific requirements and goals of the DL model.

At 524, the method 500 ends and a validation and performance assessment are carried out using a method 530 described in connection with FIG. 5B.

FIG. 5B is a flow-chart of a method 530 of various aspects of training, validating, testing, and deploying of ML and DL models in FIG. 5A according to a representative embodiment. Various aspects and details of the method 500 may be common to those described above in connection with the representative embodiments of FIGS. 1-5A. These aspects and details may not be repeated in order to avoid obscuring the presently described representative embodiments. It is emphasized that the method 500 is adapted to be carried out using a computer system such as computer system 115, or may be carried out on another computer system, with the trained and tested ML or DL model(s) being stored as executable code (instructions) in memory 130 or database 140 for execution by a processor of the controller 120. Alternatively, the trained and tested ML and DL models may be stored remotely and executed remotely as well.

At 532 the method begins.

At 534 ECG and IMU data are simultaneously gathered using ECG and IMU sensors such as described above.

At 536 the method 530 comprises data preparation is carried out. This step comprises 510˜520 described above.

At 538 a training set is gathered. The training set comprises ECG and IMU data designated for initial training of the model. Again, the training comprises training both feature-based ML models and end-to-end DL models. The feature-based ML models typically extract relevant features from the data and use them as inputs for classification or regression tasks. In contrast, the end-to-end DL models learn directly from the raw data, bypassing the need for explicit feature extraction for classification or regression. For purpose of illustration, it is assumed that the method 530 includes both ML and DL models, although it is understood that the method 530 would also apply for only ML models or DL modules.

At 540 the models are trained. Training of each of the models is carried out using a training set of data. The training set is used to teach the model how to make predictions regarding the determination of an ND or the current stage of the ND, or both. Data from the training set are provided to the model, which learns to associate the input data with the desired output. During the training of the model, the application of data augmentation and automation techniques can enhance the generalization ability of both ML and DL models, enabling them to achieve higher performance on new, unseen data. Further details of the data augmentation may be found in “Effective Data Augmentation, Filters, and Automation Techniques for Automatic 12-Lead ECG Classification Using Deep Residual Neural Networks” to An, J., Gregg, R.E. and Borhani, S. IEEE Engineering in Medicine & Biology Society (EMBC), (pp. 1283-1287) (2022), the disclosure of which is specifically incorporated herein by reference and for all purposes.

After training, at 542, the trained models are validated with a validation set from 544 to evaluate the models' performance. As shown in FIG. 5B, the models do not access the validation set during training. This ensures that the models are not overfitting to the training set. Overfitting occurs when the models learn the training set too well and as a result, the models perform poorly on data that it has not accessed previously. The validation set is a separate portion of the data that is used to fine-tune the model during the training process. It serves as an intermediate step to assess how well the model is generalizing to new, unseen data. After each training iteration or epoch, the model's performance on the validation set is evaluated by computing relevant metrics (e.g., F1 score, accuracy, precision, recall, etc.). These metrics provide insights into the model's effectiveness/accuracy and help in making decisions regarding hyperparameter tuning and model selection.

At 546 a fine-tuning sequence is carried out on the validated trained model. Specifically, in this part of the method, based on the model's performance on the validation set, adjustments can be made to the model's hyperparameters, which are settings that determine how the model learns. Examples of hyperparameters include the learning rate, batch size, number of hidden layers, and number of neurons in each hidden layer, activation functions, dropout rate, regularization parameters, or loss functions. Fine-tuning involves modifying these hyperparameters to improve the model's performance. Fine-tuning is an iterative process where different combinations of hyperparameters are tested, and the model is trained and validated multiple times until an acceptable performance level is achieved.

After fine-tuning, at 542 the model is again validated, and as needed, fine-tuning is carried out again at 546. At 550 a test set 548 of ECG and IMU data is evaluated by application of the predictive model, which may be an ML or DL classification model or an ML or DL regression model, where the classification model provides a value indicative of the detection or not of the ND under test, and the regression model to evaluates changes in the subject since previous testing. Once trained and fine-tuned using the training and validation sets described above, the model is then evaluated on a test data set that has not been used for training and validating (often referred to as held-out test data). The test data set serves as a final evaluation step to assess the model's performance on data not previously provided to the model, and provides an estimate of how well the model is likely to perform in real-world scenarios. At 552 the ML/DL models are deployed. Again, these trained and tested models are stored for execution by a processor, such as being stored in memory 130 or database 140, and executed by a processor of the controller 120.

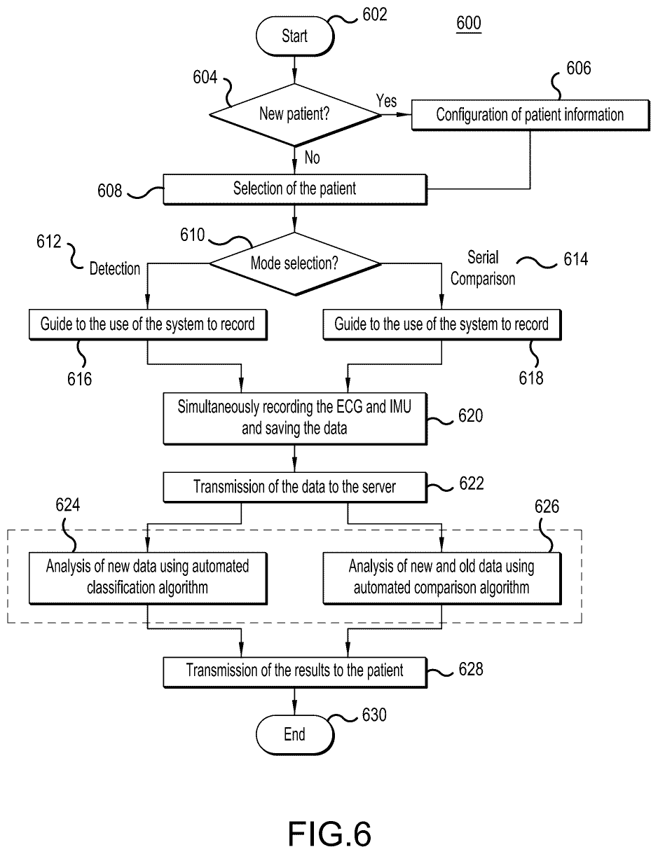

FIG. 6 is a simplified flow-diagram showing a method 600 of detecting a presence of an ND, and changes of the ND, using computational models according to a representative embodiment. Notably, the computational models of the method 600 are trained, validated, and tested according to methods 500, 530. Various aspects and details of the method 500 may be common to those described above in connection with the representative embodiments of FIGS. 1-5B. These aspects and details may not be repeated in order to avoid obscuring the presently described representative embodiments. It is emphasized that the method 600 is adapted to be carried out using a computer system such as computer system 115, or may be carried out on another computer system, with the trained and tested ML or DL model(s) being stored as executable code (instructions) in memory 130 or database 140 for execution by a processor of the controller 120. Alternatively, the trained and tested ML or DL models may be stored remotely and executed remotely as well.

At 602, the method 600 begins.

At 604, based on data input to the system (e.g., system 100), the determination is made whether the patient is a new patient, or whether the patient has undergone the determination of an ND test before.

If the answer is yes, the method proceeds to 606, and the patient's/subject's information is gathered, such as from memory 130, or database 140, or from remote storage.

If the answer is no, the method proceeds to 608 to 610 where mode selection is made. Specifically, when the desired test is to detect the existence of an ND, the method 600 proceeds to 612, whereas when the desired test is a serial comparison to measure the progression/regression of the ND, the method 600 proceeds to 614.

At 616 and 618, depending on the selected mode, the method 600 proceeds to guide the use of the system to record IMU and ECG data using IMU and ECG sensors described above. That is, at 616 the system records IMU and ECG data relating to detection and at 618 the system records IMU and ECG data relating to monitoring changes.

At 620, the method 600 proceeds to record IMU and ECG data from the subject, and save the data to memory (e.g., memory 130 or database 140).

After completion of data collection, the method proceeds to 622 where the data are transmitted for analysis by the classification or regression computational models such as described in connection with representative embodiments. As noted above, the first computational model is used to detect the presence of an ND, and the second computational model is used to determine the progression/regression of the ND for the subject.

At 624, the results of the application of the models are transmitted to the subject, such as for example, on the smart phone, watch/fitness tracker, or other communication device used in the testing as noted above.

Finally, at 630, the method 600 ends.

In the present teaching, computational models are stored as instructions to provide the machine-learning algorithm, such as a convolutional neural-network deep learning algorithm. When executed by a processor, the ML and DL algorithms (the computational models) of the representative embodiments are used to infer the presence of an ND, or the progression/regression of the ND during treatment, or both.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

Although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

1. A method of analyzing a neurodegenerative and/or a neurological disorder (ND), the method comprising:
applying an electrocardiogram (ECG) sensor and an inertial measurement unit (IMU) sensor to a subject;
gathering ECG data and IMU data for the subject;
inputting the ECG data and IMU data to a first computational model and a second computational model; and
inferring a presence of the ND based on the first computational model, or a change in the ND based on the second computational model.

2. The method of claim 1, wherein when inferring the presence of the ND, the first computational model comprises a classification model.

3. The method of claim 2, wherein the classification model comprises one of a feature-based machine learning (ML) algorithm or an end-to-end deep learning (DL) model.

4. The method of claim 1, wherein when inferring the change of the ND, the second computational model comprises regression model.

5. The method of claim 4, wherein the regression model comprises one of feature-based machine learning (ML) algorithm or an end-to-end deep learning (DL) model.

6. The method of claim 1, further comprising, after the ECG and IMU data are gathered, compiling ECG ground truth data (GTD) and IMU GTD from the subject; and
training and testing the first computational model to predict the presence of the ND.

7. The method of claim 1, further comprising, after the ECG and IMU data are gathered, compiling ground truth ECG and IMU data from the subject; and
training the second computational model to predict the change in the ND.

8. The method of claim 7, wherein IMU and ECG GTD comprises one or more features related to postural sway, comprising the elliptical area sway, entropy, fractal dimension, fractal dynamics, a Lyapunov exponent, raw ECG signals, heart rate variability (HRV), QT-interval, power spectral density (PSD).

9. A system for analyzing a neurodegenerative and/or a neurological disorder (ND), comprising:
an electrocardiogram (ECG) sensor and an inertial measurement unit (IMU) sensor configured to collect ECG data and IMU data for a subject, respectively;
a processor;
a memory that stores instructions, which when executed by the processor, cause the processor to:
gather the ECG data and the IMU data for the subject;
input the ECG data and the IMU data to a first computational model and a second computational model; and
infer a presence of the ND based on the first computational model, or a change in the ND based on the second computational model.

10. The system of claim 9, wherein when the processor infers the presence of the ND, the first computational model comprises a classification model.

11. The system of claim 10, wherein the classification model comprises one of a feature-based machine learning (ML) algorithm or an end-to-end deep learning (DL) model.

12. The system of claim 9, wherein when the processor infers the change of the ND, the second computational model comprises regression model.

13. The system of claim 12, wherein the regression model comprises one of feature-based machine learning (ML) algorithm or an end-to-end deep learning (DL) model.

14. The system of claim 9, wherein the instructions, when executed by the processor, and after the ECG and IMU data are gathered, cause the processor to:
compile ECG ground truth data (GTD) and IMU GTD from the subject; and
train and test the first computational model to predict the presence of the ND.

15. The system of claim 9, wherein the instructions, when executed by the processor, and after the ECG and IMU data are gathered, cause the processor to:
compile ECG ground truth data (GTD) and IMU GTD from the subject; and
train the second computational model to predict the change in the ND.

16. A tangible, non-transitory computer readable medium that stores instructions, which when executed by a processor, cause the processor to:
gather ECG and IMU data for a subject;
input the ECG and IMU data to a first computational model and a second computational model; and
infer a presence of a neurodegenerative and/or a neurological disorder (ND) based on a first computational model, or a change in the ND based on a second computational model.

17. The tangible, non-transitory computer readable medium of claim 16, wherein the instructions, when executed by the processor, and after the ECG and IMU data are gathered, cause the processor to:
compile ECG ground truth data (GTD) and IMU GTD from the subject; and
train the first computational model to predict the presence of the ND.

18. The tangible, non-transitory computer readable medium of claim 16, wherein the instructions, and after the ECG and IMU data are gathered, cause the processor to:
compile ECG ground truth data (GTD) and IMU GTD from the subject; and
train the second computational model to predict the change in the ND.

19. The tangible, non-transitory computer readable medium of claim 16, wherein the instructions, when executed by the processor, and after the ECG and IMU data are gathered, cause the processor to:
compile ECG ground truth data (GTD) and IMU GTD from the subject; and
train the first computational model to predict the presence of the ND.

20. The tangible, non-transitory computer readable medium of claim 16, wherein when the processor infers the presence of the ND, the first computational model comprises a classification model.

21. The tangible, non-transitory computer readable medium of claim 20, wherein the classification model comprises one of a feature-based machine learning (ML) algorithm or an end-to-end deep learning (DL) model.

22. The tangible, non-transitory computer readable medium of claim 16, wherein when the processor infers the change of the ND, the second computational model comprises regression model.

23. The tangible, non-transitory computer readable medium of claim 22, wherein the regression model comprises one of feature-based machine learning (ML) algorithm or an end-to-end deep learning (DL) model.
